(19) United States
(12) Patent Application Publication
(10) Pub. No.: US 2021/0346207 A1
(43) Pub. Date: Nov. 11, 2021

(54) FOUR-WAY STRETCH THERAPEUTIC COMPRESS ASSEMBLY, SYSTEM, AND METHOD

(71) Applicant: Marc Sheldon Stevens, Stateroad, NC (US)

(72) Inventor: Marc Sheldon Stevens, Stateroad, NC (US)

(21) Appl. No.: 17/301,911

(22) Filed: Apr. 19, 2021

Related U.S. Application Data
(60) Provisional application No. 62/704,444, filed on May 11, 2020.

(51) Int. Cl. A61F 13/00; A61F 13/53; A61F 13/514; A61F 13/02

(52) U.S. Cl. CPC A61F 13/00063; A61F 13/53; A61F 13/51401; A61F 2013/00285; A61F 2013/00119; A61F 2013/00174; A61F 2013/0028; A61F 13/0203

(57) ABSTRACT

A therapeutic compress assembly, system, and method for treating an area of a user is disclosed herein. The therapeutic compress assembly includes an elastic fabric compress and an absorbent pad. The elastic fabric compress is stretchable in a length direction and a width direction. The absorbent pad is coupled to a first side of the elastic fabric compress. The absorbent pad may be impregnated with a medication or an oil. In use, the therapeutic compress assembly is applied to the area of the user.

### CROSS-REFERENCE TO RELATED APPLICATION

This application claims the benefit of priority of U.S. provisional application No. 62/704,444, filed May 11, 2020, the contents of which are herein incorporated by reference.

### BACKGROUND OF THE INVENTION

The present invention relates to medical compresses and, more particularly, to a viscoelastic therapeutic compress impregnated with medicated materials and applied to areas requiring medical treatment.

Joint pain, inflammatory conditions, and/or musculoskeletal disorders are ailments that millions of people, just within the United States, experience every year. These ailments generally require targeted treatments to attempt to relieve any resultant aches, pain, swelling, discomfort, weakness, and more.

Conventional treatments include the use of kinesiology tape to facilitate motion by providing support, lessen pain, improve lymph transport and increase blood circulation. These conventional treatments fall short for many reasons, including because they do not allow application of various medication and/or oils while maintaining structural support. In particular, conventional products (like kinesiology tape) are designed to only stretch unidirectionally. These products lack multi-directional elasticity, which negatively impacts the overall adherence of the product to a user's skin (e.g., during physical activity such as exercising) as well as blood flow in the surrounding tissue of the treatment area. Conventional products also do not allow variability prior to application.

In light of the foregoing discussion, there is clearly a need for a therapeutic compress that may be impregnated with various medicated materials, capable of stretching axially in multiple directions, and therefore can be applied to any areas to be medically treated, without concern of loss of flexibility or restricted blood flow.

### SUMMARY OF THE INVENTION

In one aspect of the present invention, a therapeutic compress assembly for treating an area of a user is disclosed. The assembly comprises an elastic fabric compress stretchable in a length direction and a width direction. The assembly further comprises an absorbent pad coupled to a first side of the elastic fabric compress. The absorbent pad is configured to be impregnated with a medication or an oil for application to the area of the user.

In another aspect of the present invention, a therapeutic compress system for treating an area of a user is disclosed. The system comprises an elastic fabric compress stretchable in a length direction and a width direction. The system further comprises an absorbent pad coupled to a first side of the elastic fabric compress. The system further comprises a medication or an oil for application to the area of the user, with the absorbent pad being impregnated with the medication or the oil.

In yet another aspect of the present invention, a method for treating an area of a user is disclosed, with the method comprising the following steps. A therapeutic compress assembly is provided, and comprises an elastic fabric compress and an absorbent pad. The elastic fabric compress is stretchable in a length direction and a width direction. The absorbent pad is coupled to a first side of the elastic fabric compress. The absorbent pad is impregnated with a medication or an oil. The therapeutic compress assembly with the medication or the oil is applied to the area of the user.

Embodiments of the present invention allow application to the skin of various topical solutions and utilize the function of elasticity and recoil in the compress for better adherence and improved vascularity/blood flow to the surrounding tissue. Embodiments of the present invention are designed with a central absorbent material to hold and time-release oils, or other customizable mixtures, and are not pretreated. The material utilized is unique for the fabric of the compress, and advantageously permits elastic recoil with stretch in all directions to be applied to the skin.

These and other features, aspects and advantages of the present invention will become better understood with reference to the following drawings, description and claims.

### BRIEF DESCRIPTION OF THE DRAWINGS

FIG. 1 is a perspective view of a first embodiment of the present invention;

FIG. 2 is an exploded view of the first embodiment of the present invention;

FIG. 3 is a top plan view of the first embodiment of the present invention;

FIG. 4 is a top plan view of a second embodiment of the present invention, showing an alternate absorbent pad configuration;

FIG. 5 is a top plan view of a third embodiment of the present invention, showing another alternate absorbent pad configuration;

FIG. 6 is a top plan view of a fourth embodiment of the present invention, showing yet another alternate absorbent pad configuration; and

FIG. 7 is a perspective view of the first embodiment and the fourth embodiment of the present invention, in use.

### DETAILED DESCRIPTION OF THE INVENTION

The following detailed description is of the best currently contemplated modes of carrying out exemplary embodiments of the invention. The description is not to be taken in a limiting sense, but is made merely for the purpose of illustrating the general principles of the invention, since the scope of the invention is best defined by the appended claims.

Broadly, one embodiment of the present invention provides a therapeutic compress assembly for placement on an area of a user including an elastic fabric stretchable lengthwise and widthwise and an absorbent material coupled to a side of the viscoelastic fabric. The absorbent material is configured to be impregnated with a medication or an oil. In use, the therapeutic compress assembly is applied to the area of the user.

Further embodiments of the present invention pertain to a structural support and medication and essential oil delivery system and corresponding method. The present invention allows application of various medication/oils while maintaining support structurally for the area on the body being treated. In accordance with certain aspects of the present invention, the compress is formed from a viscoelastic fabric, and, in certain preferred embodiments, may be embodied as a lightweight breathable nylon. In this application, viscoelastic refers to a material that stretches and recovers in every direction (e.g., lengthwise and widthwise), which is also referred to in the art as four-way stretch.

For example, the lightweight breathable nylon may be tricot fabric or elastane (also referred to as spandex) and comprise various amounts thereof in combination with the nylon. Fabrics, like the aforementioned ones, exhibit the properties of being capable of stretching and recovering in both width and length directions. For example, in certain embodiments, the tricot fabric may stretch up to 25% of its length and width, and the elastane fabric may stretch up to 75% to 100% of its length and width. However, these fabrics have never been applied in the medical compress field prior to the present invention, which addresses the previously described shortcomings of the prior art, specifically the lack of multi-directional elasticity. This multi-directional elasticity is of particular use for adherence to foot, ankle, shoulder, and elbow regions, for example.

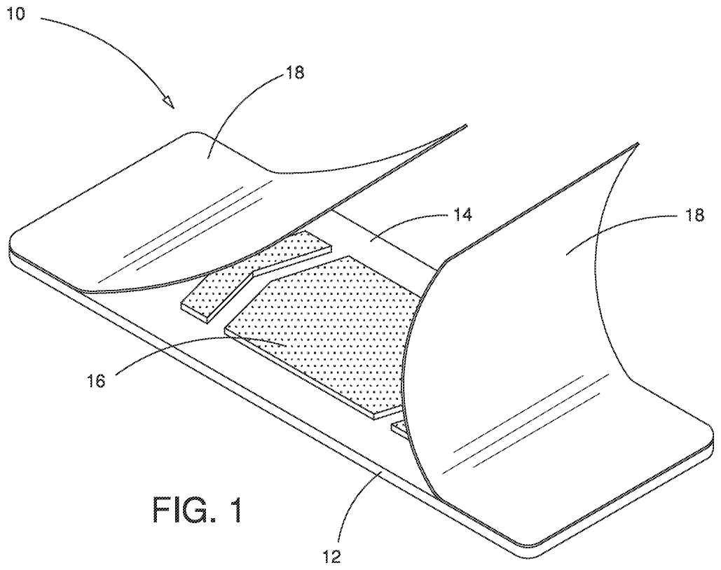

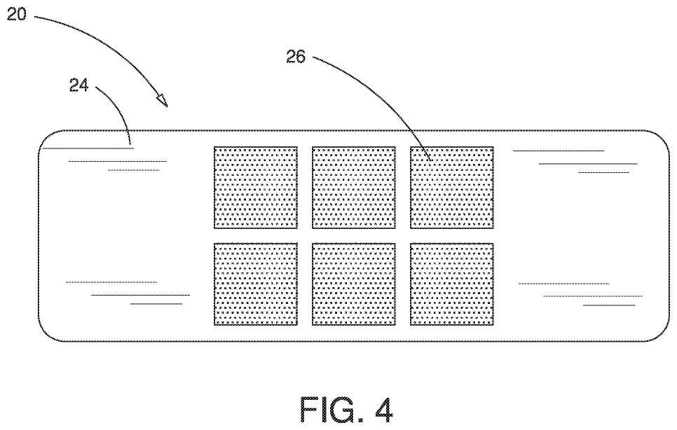

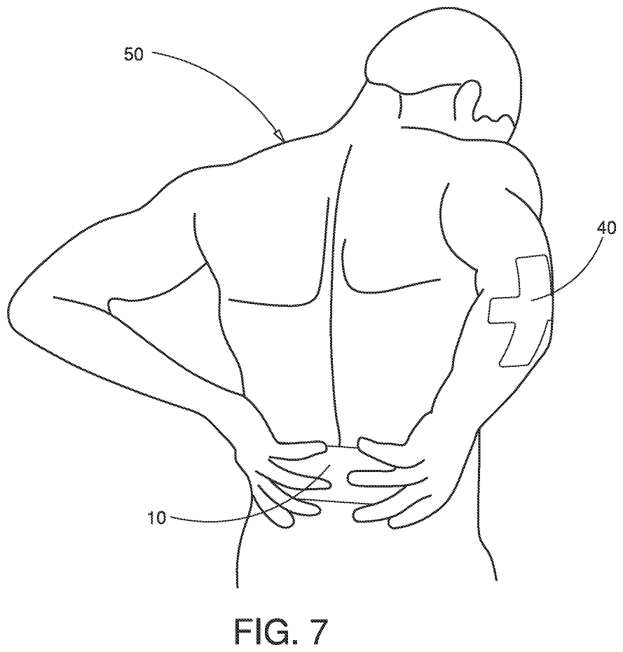

Referring now to FIG. 1, a four-way stretch therapeutic compress assembly 10 includes an elastic/viscoelastic (i.e., four-way stretch) compress 12, an impermeable backing 15, an absorbent pad 16, and a removable protective film 18. In combination, these elements form a medication-infusible support structure. As shown in FIGS. 1-7, in certain embodiments, the compress 12 may have a substantially rectangular profile (e.g., FIGS. 1-5), while in a fourth embodiment of the therapeutic compress assembly 40, the compress 44 may be cross-shaped. As would be readily apparent to those with skill in the art, various compress shapes and sizes may be utilized in accordance with the present invention. Advantageously, the four-way stretch material of the present invention facilitates improved adherence of the compress assembly 10 to a user's skin (e.g., FIG. 7), as well as improving blood flow in the surrounding tissue of the treatment area and permitting wider ranges of motion without the compress assembly 10 becoming dislodged.

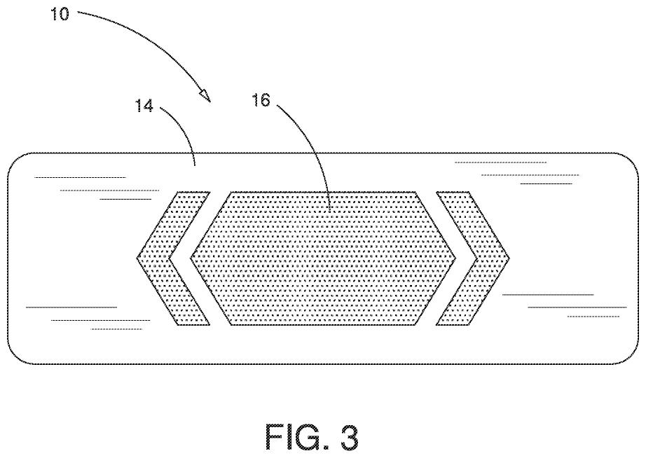

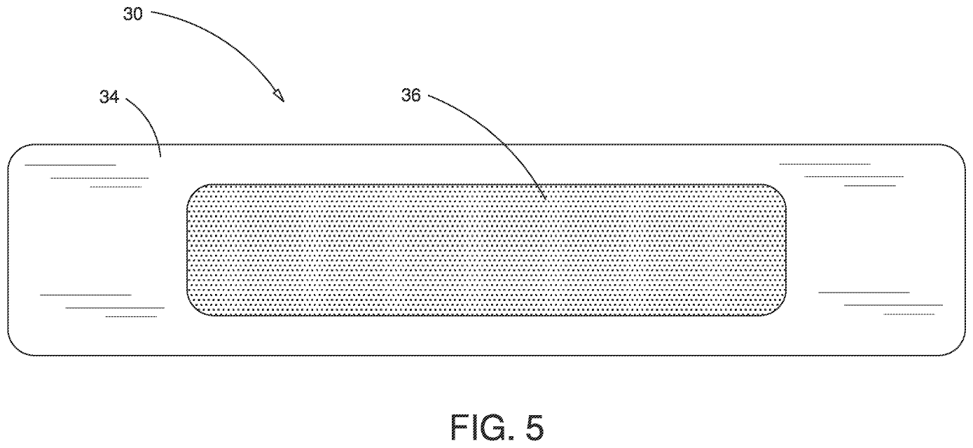

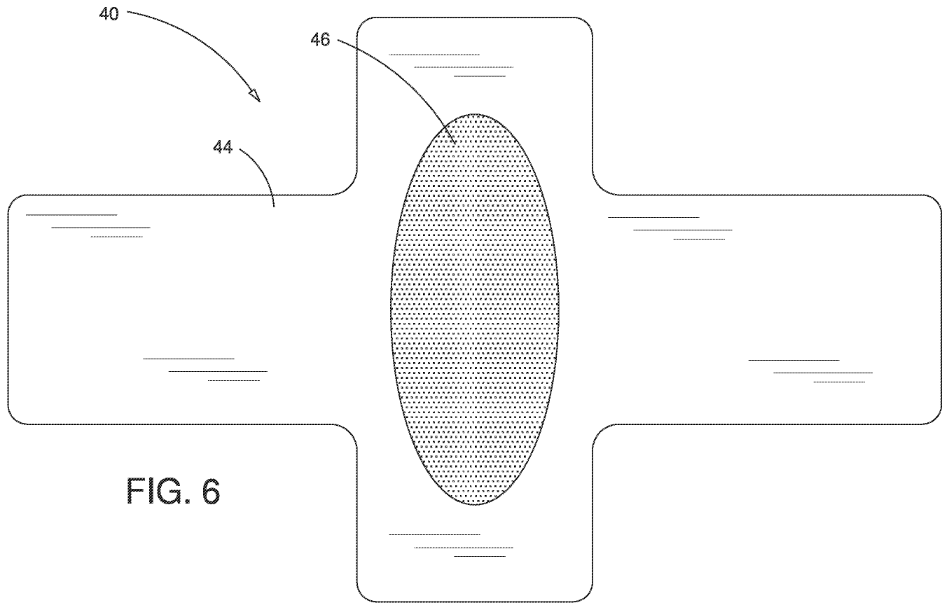

The absorbent pad 16 is secured, e.g., by sewing, to the compress 12, may be impregnated with various medications and/or essential oils prior to use, and may be formed in various pattern configurations (see FIGS. 3-6, for example). While not explicitly called out in FIGS. 3-6, it should be understood that the appearance of the compress assemblies is representative of how they also appear when impregnated with the medication(s) and/or oil(s). As shown in FIGS. 1-3, the absorbent pad 16 may be formed in a plurality of pieces, with a first embodiment resembling the look of sergeant stripes (FIGS. 1-3), and a second embodiment 20 including a plurality of square/rectangular-shaped pads 26 disposed on the compress 24. The utilization of different cells or pads to form the pad portion allows for different oils, topicals, or other medications on each pad, as well as different mixtures. Further, a grid-like pattern of pads 16 improves oil activation and significantly reduces evaporation from the skin, allowing the oils and/or ointments to be more effective over a longer period of time. However, it will be appreciated by those with skill in the art that the pad may also be formed as a singular structure, such as a substantially rectangular shape 36, as shown in a third embodiment 30 in FIG. 5, or an oval shape 46, as shown in the fourth embodiment 40 in FIG. 6. Further, in certain embodiments, the absorbent pad 16 may be formed from medical grade foam.

The impermeable backing 15 is disposed between the elastic compress 12 and absorbent pad 16 to keep the medication/oils contained to the absorbent pad so that treatment can most effectively be delivered to a target area/region on a patient. By doing so, less medications/oils are wasted and an optimal saturation of the absorbent pads against a user's skin is maintained. It will be appreciated by those with skill in the art that the impermeable backing 15 may be sized accordingly, as shown, to only separate the absorbent pad 16 and the four-way stretch compress 12, leaving the remainder of the four-way stretch compress 12 uncovered. Alternatively, the impermeable backing 15 may cover a larger region than just where the absorbent pad 16 is disposed.

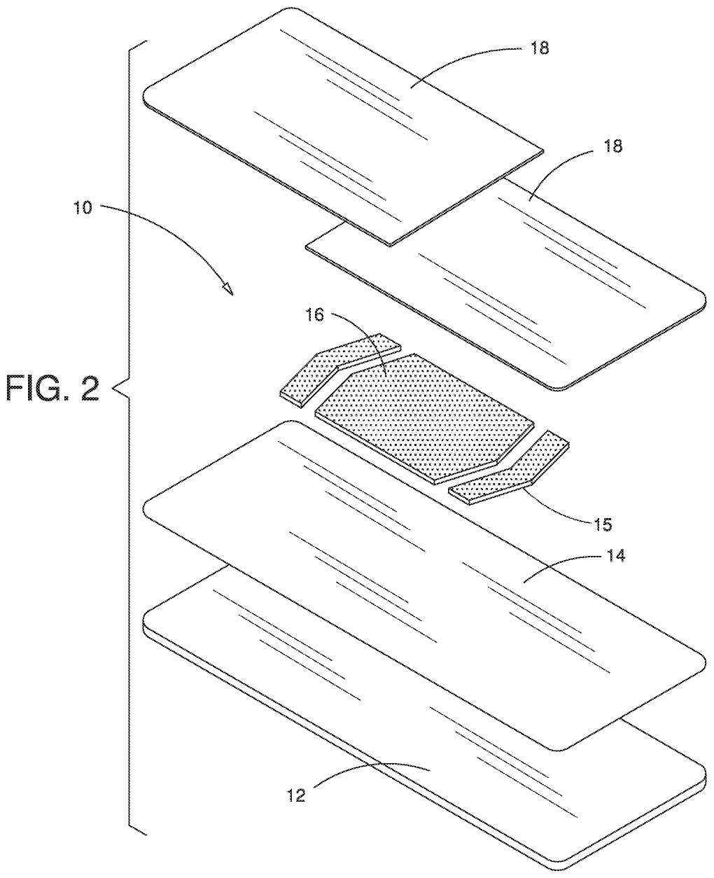

To secure the compress 12 to a body part/affected area of a patient/user 50, a non-traumatic adhesive 14 is applied in a pattern, such as a diamond pattern, to the compress 12. It will be appreciated by those with skill in the art that any appropriate pattern of adhesive 14 may be used in accordance with the present invention. As shown in FIG. 2, the adhesive 14 may span a length of the compress 12. In certain embodiments, the adhesive 14 may be a pressure-sensitive acrylic, making it gentle to the skin. Embodiments that utilize a pressure sensitive acrylic are ideal for users with sensitive skin, such as pediatric and geriatric users.

In use, as shown in FIG. 7, one or more compress assemblies 10, 40 provide support to the user 50 while the absorbent pad(s) 16, 46 release medication/oils into the tissues. It should be understood, of course, that while the means for delivering the medication/oils has been described as a pad, any suitable material and combinations thereof may be used without departing from the spirit and scope of the present invention.

A method of making the present invention may include the following. The absorbent material/pad 16 is woven to a compress 12, with an impermeable backing 15 being disposed between the compress and absorbent pad/material. The backing 15 may be sized such that it has substantially the same area as the pad 16. Medication/oils can then be applied either prior to packaging or later when unpacked and before use. A removeable protective film 18 may be adhered to the side of the compress 12 including the pad 16 to keep that portion sterile. Further, a method of using the present invention may include the following. The compress 12 and absorbent pad/material 16 with medication/oils is applied to a patient's affected area to relieve pain and to aid with improved function/range of motion. Embodiments of the present invention may also be used to treat inflammation or give other health support.

Embodiments of the present invention help in the management of injury and pain of the musculoskeletal and integumentary system secondary to trauma or degenerative changes, i.e., arthritis, tendonitis and other inflammatory conditions. Embodiments of the present invention may be used to cover wounds, help with diabetes, and cushion surfaces from abrasive sources. In general, embodiments of the present invention teach the principle of customizable oils or mixtures that can be applied to a pad integrated into an elastic, adherent fabric that can be elongated in all directions. Embodiments thereby improve recoil in the surrounding tissues, with the goal of improved vascularity and improved absorption of the medication/oils. Because embodiments of the present invention stretch four ways, not two, blood supply with isotropic properties is increased four-fold. Further, in athletes, embodiments of the present invention improve vascularity which strengthens the musculoskeletal system's ability to avoid injury, while adhering to the athlete's skin, even during strenuous exercise. In summary, embodiments of the present invention offer improved adherence, improved vascularity, and customizability compared to the prior art.

It should be understood, of course, that the foregoing relates to exemplary embodiments of the invention and that modifications may be made without departing from the spirit and scope of the invention as set forth in the following claims. It will also be appreciated that, while the first embodiment was the embodiment largely described in the foregoing disclosure, the other embodiments function in the same way, unless otherwise noted (e.g., plural pads versus a singular pad).

In the following claims, any labelling of elements, limitations, steps, or other parts of a claim (for example, first, second, etc., (a), (b), (c), etc., or (i), (ii), (iii), etc.) is only for purposes of clarity, and are not to be interpreted as suggesting any sort of ordering or precedence of the claim parts so labelled. If any such ordering or precedence is intended, it will be explicitly recited in the claim or, in some instances, it will be implicit or inherent based on the specific content of the claim. To further aid the USPTO and any readers of any patent issued on this application, it is additionally noted that there is no intent any of the appended claims to invoke paragraph (f) of 35 U.S.C. § 112 as it exists on the date of filing hereof unless the words “means for” or “step for” are explicitly used in the particular claim.

What is claimed is:

1. A therapeutic compress assembly for treating an area of a user, the assembly comprising:
an elastic fabric compress stretchable in a length direction and a width direction; and
an absorbent pad coupled to a first side of the elastic fabric compress, the absorbent pad being configured to be impregnated with a medication or an oil for application to the area of the user.

2. The therapeutic compress assembly of claim 1, wherein the elastic fabric compress is recoverable in the length direction and the width direction.

3. The therapeutic compress assembly of claim 1, wherein the elastic fabric compress is a lightweight, breathable material.

4. The therapeutic compress assembly of claim 3, wherein the lightweight, breathable material comprises a tricot fabric.

5. The therapeutic compress assembly of claim 3, wherein the lightweight, breathable material comprises elastane.

6. The therapeutic compress assembly of claim 1, further comprising an impermeable backing disposed between the elastic fabric compress and the absorbent pad, the impermeable backing being configured to prevent the medication or the oil from seeping through to the elastic fabric.

7. A therapeutic compress system for treating an area of a user, the system comprising:
an elastic fabric compress stretchable in a length direction and a width direction;
an absorbent pad coupled to a first side of the elastic fabric compress; and
a medication or an oil for application to the area of the user, with the absorbent pad being impregnated with the medication or the oil.

8. The therapeutic compress system of claim 7, wherein the elastic fabric compress is recoverable in the length direction and the width direction.

9. The therapeutic compress system of claim 7, wherein the elastic fabric compress is a lightweight, breathable material.

10. The therapeutic compress system of claim 9, wherein the lightweight, breathable material comprises a tricot fabric.

11. The therapeutic compress system of claim 9, wherein the lightweight, breathable material comprises elastane.

12. The therapeutic compress system of claim 7, further comprising an impermeable backing disposed between the elastic fabric compress and the absorbent pad, the impermeable backing being configured to prevent the medication or the oil from seeping through to the elastic fabric.

13. A method for treating an area of a user comprising:
providing a therapeutic compress assembly, the therapeutic compress assembly comprising:
an elastic fabric compress stretchable in a length direction and a width direction; and
an absorbent pad coupled to a first side of the elastic fabric compress; and
impregnating the absorbent pad with a medication or an oil; and
applying the therapeutic compress assembly with the medication or the oil to the area of the user.

14. The method of claim 13, wherein, prior to applying the therapeutic compress assembly, an adhesive is applied to the first side of the elastic compress assembly.
